(19) United States
(12) Patent Application Publication
(10) Pub. No.: US 2024/0168432 A1
(43) Pub. Date: May 23, 2024

(54) METHOD AND SYSTEM FOR CONTROLLING BIOLOGICAL PROCESSES

(71) Applicants: David Hanson, Hong Kong (HK); Dominic Man-Kit Lam, Hong Kong (HK); Olivia Yee-Yee Lam, Los Angeles, CA (US)

(72) Inventors: David Hanson, Hong Kong (HK); Dominic Man-Kit Lam, Hong Kong (HK); Olivia Yee-Yee Lam, Los Angeles, CA (US)

(21) Appl. No.: 17/978,896

(22) Filed: Nov. 1, 2022

(51) Int. Cl. G03H 1/08; G03H 1/00; G16H 15/00; G16H 30/40; G16H 50/20; G16H 50/30

(52) U.S. Cl. CPC G03H 1/0808; G03H 1/0005; G16H 15/00; G16H 30/40; G16H 50/20; G16H 50/30

(57) ABSTRACT

A method for controlling informatics and biological processes is disclosed. The method may include projecting at least one portion of a body of a user with coherent holographic light waves by use of a coherent light source. The method includes generating a plurality of images based on the projection of the at least one portion of the body of the user with the coherent holographic light waves. The method includes analyzing the generated plurality of images to determine at least one of: biological information of the body of the user, or information states of a nervous system associated with the user. The method includes transmitting one or more signals to at least neurons in the body of the user based on the analyzed plurality of images for control of one or more biological processes associated with the user.

### TECHNICAL FIELD

This disclosure relates generally to holographic imaging techniques, and more particularly to method and system for controlling informatics and biological processes within humans and other organisms using the holographic imaging techniques.

### BACKGROUND

With the advent of immersive technologies such as Artificial intelligence and reinforcement learning, there has been increasing emphasis on developing imaging techniques in healthcare. Conventional imaging techniques such as Magnetic resonance imaging (MRI), computed tomography scan (CT scan), and positron emission tomography (PET) scan are less useful in detecting irregularities in human body at a neuron level. Further, conducting the MRI may be unsuccessful in a few users, as there are a few restrictions associated with the MRI. For example, due to a surgical procedure, a person may have a metal plate in their head. The conducting of MRI on such a person may be difficult as the MRI utilizes a very strong magnet that may attract the metal plate on or inside the human body. Moreover, the CT scans and the PET scans are costly, non-portable and require a huge-sized equipment. Further, chemicals used in these scans for analyses, may have some side effects on the users.

Furthermore, conventional Brain-computer interfaces (BCIs) provides a real-time bidirectional link between human brains and the computer interfaces. However, the Bas may be unable to provide accurate information associated with informatics and biological processes of the human brain. Thus, there is still a need of an improved, non-invasive, safe and an easy-to-implement method for controlling the informatics and biological processes within the humans and other organisms.

### SUMMARY

In one embodiment, a method for controlling informatics and biological processes is disclosed. The method may include projecting at least one portion of a body of a user with coherent holographic light waves by use of a coherent light source. The method may further include generating a plurality of images based on the projection of the at least one portion of the body of the user with the coherent holographic light waves. The method may further include analyzing the generated plurality of images to determine at least one of: biological information of the body of the user, or information states of a nervous system associated with the user. The method may further include transmitting one or more signals to at least neurons in the body of the user based on the analyzed plurality of images for control of one or more biological processes associated with the user. The one or more signals are transmitted based on the projection of the coherent holographic light waves.

In another embodiment, a system for controlling informatics and biological processes is disclosed. The system may include a processor and a memory communicatively coupled to the processor. The memory stores a plurality of processor-executable instructions which upon execution by the processor cause the processor to project at least one portion of a body of a user with coherent holographic light waves by use of a coherent light source. The plurality of processor-executable instructions, upon execution by the processor, may further cause the processor to generate a plurality of images based on the projection of the at least one portion of the body of the user with the holographic light waves. The plurality of processor-executable instructions, upon execution by the processor, may further cause the processor to analyze the generated plurality of images to determine at least one of: biological information of the body of the user, or information states of a nervous system associated with the user. The plurality of processor-executable instructions, upon execution by the processor, may further cause the processor to transmit one or more signals to at least neurons in the body of the user based on the analyzed plurality of images for control of one or more biological processes associated with the user. The one or more signals are transmitted based on the projection of the coherent holographic light waves.

In yet another embodiment, a non-transitory computer-readable medium storing computer-executable instructions for controlling informatics and biological processes is disclosed. The computer-executable instructions may be configured for projecting at least one portion of a body of a user with coherent holographic light waves by use of a coherent light source. The computer-executable instructions may be further configured for generating a plurality of images based on the projection of the at least one portion of the body of the user with the holographic light waves. The computer-executable instructions may be further configured for analyzing the generated plurality of images to determine at least one of: biological information of the body of the user, or information states of a nervous system associated with the user. The computer-executable instructions may be further configured for transmitting one or more signals to at least neurons in the body of the user based on the analyzed plurality of images for control of one or more biological processes associated with the user. The one or more signals are transmitted based on the projection of the coherent holographic light waves

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of a system for controlling informatics and biological processes, in accordance with an embodiment;

FIG. 2 is a functional block diagram of the system for controlling informatics and biological processes, in accordance with an embodiment;

FIG. 3 is a flow diagram of method steps for determining biological information and information states, in accordance with an embodiment;

FIG. 4 is a flow chart for displaying feedback associated with informatics and biological processes, in accordance with an embodiment;

FIG. 5 is a flow chart depicting a method for controlling informatics and biological processes, in accordance with an embodiment; and

FIG. 6 a block diagram of an examplary computer system, in accordance with an embodiment.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims. Additional illustrative embodiments are listed below.

One or more techniques are disclosed that seek to provide a solution to the above-mentioned challenges faced by the technologies of the prior art, by use of coherent holographic light waves to generate holographic images or holograms. A hologram is a three-dimensional image reproduced from a pattern of interference produced by a split coherent beam of radiation. In general, holography provides communication for humans due to the extensive capability of three-dimensional (3D) visualization of a body of a patient with high resolution. It is used to simplify the procedure of complex treatments. The holograms created by use of the coherent holographic light waves may be utilized in, for example, clinical applications by the healthcare professionals.

In addition, the techniques of the present disclosure are based on deep reinforcement learning algorithm applied to the generated holographic images or videos. The generated holographic videos may be analyzed using one or more artificial intelligence or machine learning algorithms such as deep reinforcement learning algorithm in order to determine biological information and information states of a user, such as a human being and further generate medical reports for perusal of a medical professional or the user. Further, the techniques may analyze the determined biological information and the information states of the user to control one or more biological processes associated with the user.

Further, these techniques may be helpful in providing a human-computer interface that may be intuitive and predictable. The human-computer interface may allow the human to interact with the computer. The human-computer interface enables the computer to provide recommendations or suggestions to the human based on the determined biological information and information states of the human.

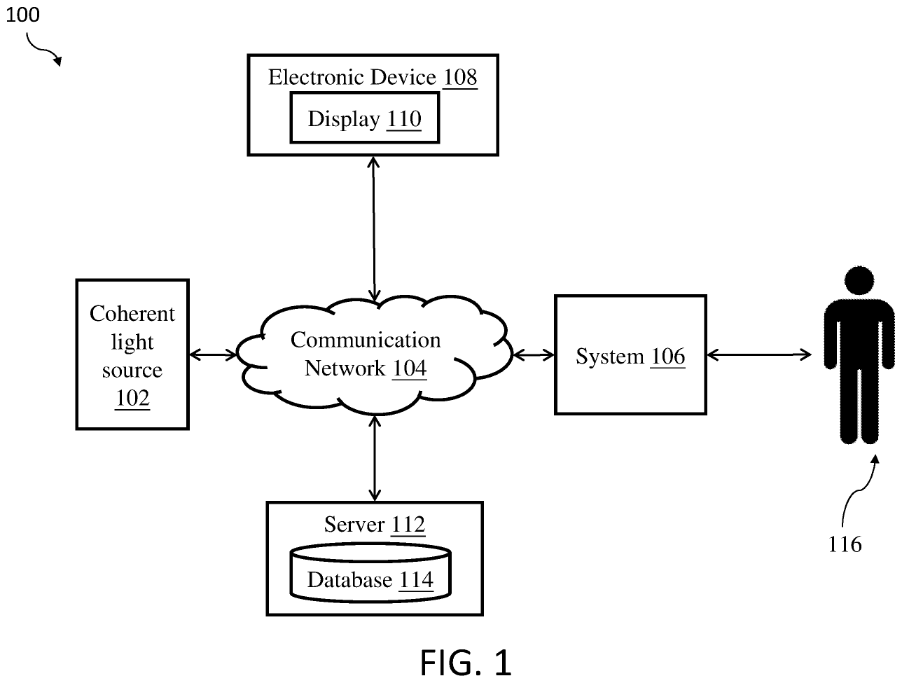

In one embodiment, a block diagram 100 of a system 106 for controlling informatics and biological processes of a user is illustrated in FIG. 1, in accordance with an embodiment. The block diagram 100 may include a coherent light source 102, a communication network 104, the system 106, an electronic device 108, a server 112, a database 114, and the user 116.

The coherent light source 102 may be utilized to project coherent holographic light waves on at least a portion of a body of the user 116. In general, the coherent light source 102 emits a light wave that may have a same frequency and wavelength and a constant phase difference. The coherent light source 102 may form sustained interference patterns during an occurrence of superimposition of light waves. In an embodiment, the coherent light source 102 may include, but may not be limited to, a laser light. In general, the laser light may utilize a stimulated emission phenomenon to generate a highly coherent light. The highly coherent light from the laser light may be a parallel, monochromatic light that may have an unbroken wave chain.

The system 106 may be configured to project the at least one portion of the body of the user 116 with the coherent holographic light waves by use of the coherent light source 102. The system 106 may be further configured to generate a plurality of images based on the projection on the at least one portion of the body of the user 116. The plurality of images may correspond to images of the at least one portion of the body of the user 116. In one or more embodiments, the user 116 may be a human being.

In an embodiment, the at least one portion of the body of the user 116 may be associated with at least one of an organ of the user 116, a soft tissue of the user 116, a bone of the user 116, or a brain of the user 116. In an example, the user 116 may be a patient who may need medical assistance. In another example, the user 116 may be an individual with at least one disability such as Parkinson's, Alzheimer's, Amyotrophic lateral sclerosis (ALS), paralysis, post-coma unresponsiveness, and the like.

Further, the system 106 may be configured to analyze the generated plurality of images to determine at least one of biological information of the body of the user 116, or information states of a nervous system of the user 116. In an embodiment, the determined biological information of the body of the user 116 is associated with at least one of a probability of occurrence of stroke, information related to heart disease, information related to asthma, or other health conditions of the user 116. In addition, the determined information states of the nervous system of the user 116 is associated with at least one of a mental state, thoughts, an emotion, an intention, motor actions, a speech, a dream, a blood flow, a neural activity, a molecular activity, or pathological information related to the user 116.

The determined biological information of the body of the user 116 and the information states of the nervous system of the user 116 is displayed on a display 110 of the electronic device 108 associated with the system 106 via the communication network 104. The electronic device 108 includes, but may not be limited to, laptop, mobile phone, smartphone, desktop computer, personal digital assistant (PDA), palmtop, and tablet. The display 110 of the electronic device 108 corresponds to a screen for visual representation of any data/information, the plurality of images, the determined biological information of the body of the user 116 or the information states of the nervous system of the user 116. Examples of the display 110 may include, but may not be limited to, a liquid crystal display (LCD), a light emitting diode (LED) display, an organic light emitting diode (OLED) display, an active-matrix organic light emitting diode (AMOLED) display, and the like.

The system 106 may be further configured to transmit one or more signals to at least neurons in the body of the user 116 based on the analyzed plurality of images for control of one or more biological processes associated with the user 116. The one or more signals may be transmitted based on the projection of the coherent holographic light waves. The coherent light source 102 may be utilized for the transmission of the one or more signals to at least neurons in the body of the user 116.

The system 106 is connected to the server 112 via the communication network 104 using a plurality of methods. The server may include the database 114. The plurality of methods used to provide network connectivity to the system 106 may include communication techniques such as a second-generation cellular network (2G), a third generation of cellular technology (3G), a fourth-generation wireless technology (4G), a fifth-generation wireless technology (5G), and the like. In general, the server 112 is a computer program or device that provides functionality for other programs or devices.

The server 112 provides various functionalities, such as sharing data or resources among multiple clients, or performing computation for a client. However, those skilled in the art would appreciate that the system 106 may be connected to a greater number of servers. Furthermore, it may be noted that the server 112 may include the database 114 or may be communicatively coupled to an external database. The server 112 may be configured to handle each operation and task performed by the system 106. In one embodiment, the server 112 is located remotely.

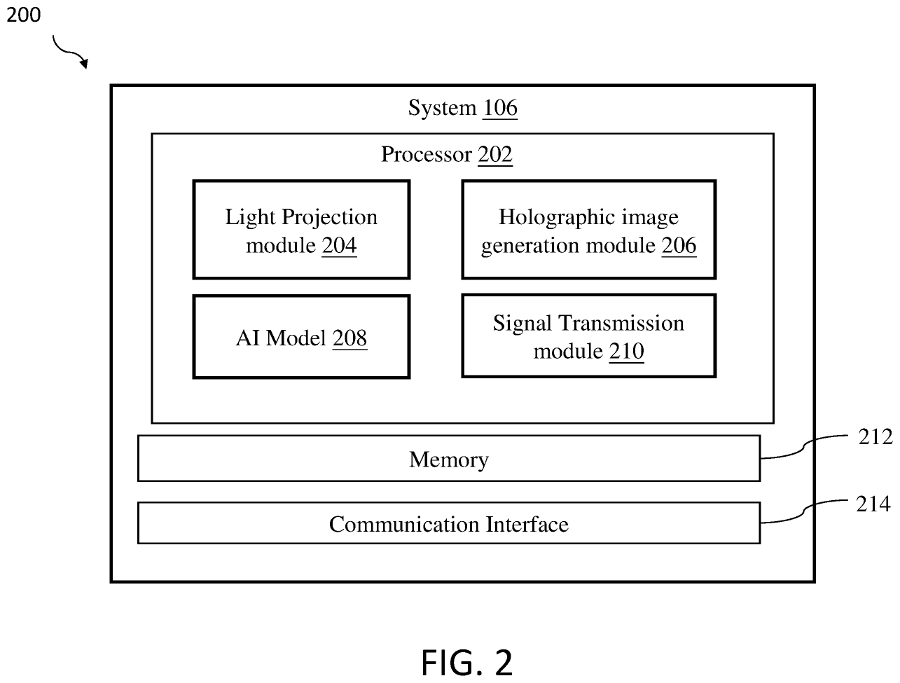

In some embodiments, the server 112 is associated with an administrator. In addition, the administrator manages the different components associated with the system 106. The administrator is any person or individual who monitors the working of the system 108 and the server 112 in real-time. The administrator monitors the working of the system 108 and the server 112 through a computing device. Examples of the computing device may include, but are not limited to, a laptop, a desktop a computer, tablet and a personal digital assistant. In addition, the database 114 may be configured to store data associated with the plurality of images, the holographic videos, the determined biological information of the body of the user 116 or the information states of the nervous system associated with the user 116, and so forth. The database 114 organizes the data using model such as relational models or hierarchical models. The database 114 also stores data provided by the administrator. Details of components of the system 106 are further provided, for example, in FIG. 2.

The system 106, the coherent light source 102, the electronic device 108 and the server 112 may be communicatively coupled with each other via the communication network 104. The communication network 104 may be a wired network or a wireless network and the examples may include the Internet, Wireless Local Area Network (WLAN), Wi-Fi, Long Term Evolution (LTE), Worldwide Interoperability for Microwave Access (WiMAX), General Packet Radio Service (GPRS) and the like. In an embodiment, the communication network 104 is a combination of the wireless and wired network. The communication network 104 may include a set of channels. Each channel of the set of channels supports a finite bandwidth. The finite bandwidth of each channel of the set of channels is based on capacity of the communication network 104.

Referring now to FIG. 2, a functional block diagram 200 of the system 106 of FIG. 1 for controlling informatics and biological processes is illustrated, in accordance with an embodiment. The system 106 may include a processor 202, a memory 212, and a communication interface 214. In some embodiments, the processor 202 may include a light projection module 204, a holographic image generation module 206, an artificial intelligence (AI) model 208, and a signal transmission module 210.

The processor 202 may be embodied in a number of different ways. For example, the processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 202 may include one or more processing cores configured to perform independently. A multi-core processor may enable multiprocessing within a single physical package. Additionally, or alternatively, the processor 202 may include one or more processors configured in tandem via the bus to enable independent execution of instructions, pipelining and/or multithreading.

The light projection module 204 may be configured to project the at least one portion of the body of the user 116 with the coherent holographic light waves by use of the coherent light source 102. The light projection module 204 may utilize different lights of different wavelengths and frequencies for different portions of the body of the user 116. For example, if retina of an eye of the user 116 is being used as a pathway to send a coherent light wave as an input, in such a case the light projection module 204 is configured to utilize visible range of frequencies of the light wave. In another example, if a different tissue of the body of the user 116 is targeted, such as cancer cells in a leg portion of the user 116, in such a case the light projection module 204 may utilize other the coherent holographic light waves of different wavelengths and frequencies. Once the light wave reaches the tissue, the generation of the plurality of images may begin.

The holographic image generation module 206 may be configured to utilize the coherent holographic light waves projected by the coherent light source 102 to generate the plurality of images of the at least one portion of the body of the user 116. In addition, in some embodiments, the holographic image generation module 206 is configured to generate a holographic video based on the plurality of images. Further, the generated plurality of images or the generated holographic video may be utilized by the AI model 208 for determining at least one of the biological information of the body of the user 116, or the information states of the nervous system associated with the user 116.

The AI model 208 may be configured to analyze the generated plurality of images or the generated holographic video to determine at least one of the biological information of the body of the user 116, or the information states of the nervous system associated with the user 116. In an example, the AI model 208 is configured to read intentions, or thoughts of the user 116 based on the analyzed plurality of images and the holographic video. Further, the system 106 may interact with the user 116 based on the read intentions or thoughts of the user 116 with facilitation of the communication interface 214 of the system 106.

The AI model 208 may further analyzes the generated plurality of images and the holographic video by use of at least one of machine learning algorithms related to deep reinforcement learning, cognitive architectures and the like. Generally, deep reinforcement learning is a category of machine learning and artificial intelligence where intelligent machines may learn from their actions similar to the way humans learn from experience. In addition, cognitive architecture may utilize structures of human mind and their working altogether to manage intelligent behavior in any complex environment. In an exemplary embodiment, the AI model 208 utilizes the deep reinforcement learning and cognitive architectures to analyze the generated plurality of images and the holographic video in order to train the system 106 for generating feedback associated with the determined at least one of the biological information of the body of the user 116, or the information states of the nervous system associated with the user 116. The generated feedback is displayed to the user 116 via the display 110 of the electronic device 108. In an example, the generated feedback corresponds to at least one of stressed mind, tiredness detected, high pulse rate, slow pulse rate, blood pressure range, and the like. In another example, the generated feedback may include “no complication detected”, “no disease detected”, “no disability detected”, and the like. Furthermore, the AI model 208 may be configured to create medical reports associated with the user 116 based on at least one of the determined biological information of the body of the user 116 and the information states of the nervous system of the user 116 and the generated feedback.

The signal transmission module 210 may be configured to transmit one or more signals to at least neurons in the body of the user 116 based on the analyzed plurality of images for controlling informatics and one or more biological processes associated with the user 116. The one or more signals are transmitted based on the projection of the coherent holographic light waves. It should be noted that transmitting the one or more signals to the at least neurons in the body of the user 116 may further include an interference of the coherent holographic light waves transmitted as the one or more signals, to transfer energy associated with the coherent holographic light waves projected on the at least one portion of the body of the user 116 at a particular time. In addition, transmitting the one or more signals to the at least neurons in the body of the user 116 may further include controlling an interference of the coherent holographic light waves transmitted as the one or more signals, to dampen energy associated with the coherent holographic light waves projected to the at least one portion of the body of the user 116 at a particular time.

In an example embodiment, the processor 202 may be in communication with the memory 212. The memory 212 may store instructions that, when executed by the processor 202, cause the processor 202 to control the informatics and the biological processes in the user 116 as discussed in greater detail in FIG. 2 to FIG. 6. The memory 212 may be a non-volatile memory or a volatile memory. Examples of non-volatile memory, may include, but are not limited to a flash memory, a Read Only Memory (ROM), a Programmable ROM (PROM), Erasable PROM (EPROM), and Electrically EPROM (EEPROM) memory. Examples of volatile memory may include, but are not limited to Dynamic Random-Access Memory (DRAM), and Static Random-Access memory (SRAM). The memory 212 may also store various data (e.g. plurality of images, etc.) that may be captured, processed, and/or required by the system 106.

In an embodiment, the system 106 may not be limited to healthcare domain. The system 106 may be utilized in vehicles to monitor a driver's state of mind while driving the vehicle in real time. For example, the system 106 may determine that the driver is sleepy, based on the informatics and the biological processes in the user 116. (The determination here may be performed by similar means as explained above). The system 106 may transmit one or more signals to central nervous system of the driver through a communication device associated with the system 106 and the driver. These signals enable the driver to stay alert while driving the vehicle. The system 106 may send notifications or warnings to the communication device of the driver to stay alert in order to avoid any accident.

In some embodiments, the processor 202 may be configured to provide Internet-of-Things (IoT) related capabilities to the user 116 of the system 106. The IoT related capabilities may in turn be used to provide smart medical assistive solutions by determining biological information of the body of the user 116 and the information states of the nervous system of the user. The system 106 may be accessed using the communication interface 214. The communication interface 214 may provide an interface for accessing various features and data stored in the system 106. In addition, the communication interface 214 may provide an interface for controlling informatics and biological processes in the user 116.

The communication interface 214 may comprise input interface and output interface for supporting communications to and from the system 106 or any other component with which the system 106 may communicate. The communication interface 214 may be any means such as a device or circuitry embodied in either hardware or a combination of hardware and software that is configured to receive and/or transmit data to/from a communications device in communication with the system 106. In this regard, the communication interface 214 may include, for example, an antenna (or multiple antennae) and supporting hardware and/or software for enabling communications with a wireless communication network. Additionally, or alternatively, the communication interface 214 may include the circuitry for interacting with the antenna(s) to cause transmission of signals via the antenna(s) or to handle receipt of signals received via the antenna(s). In some environments, the communication interface 214 may alternatively or additionally support wired communication. As such, for example, the communication interface 214 may include a communication modem and/or other hardware and/or software for supporting communication via cable, digital subscriber line (DSL), universal serial bus (USB) or other mechanisms.

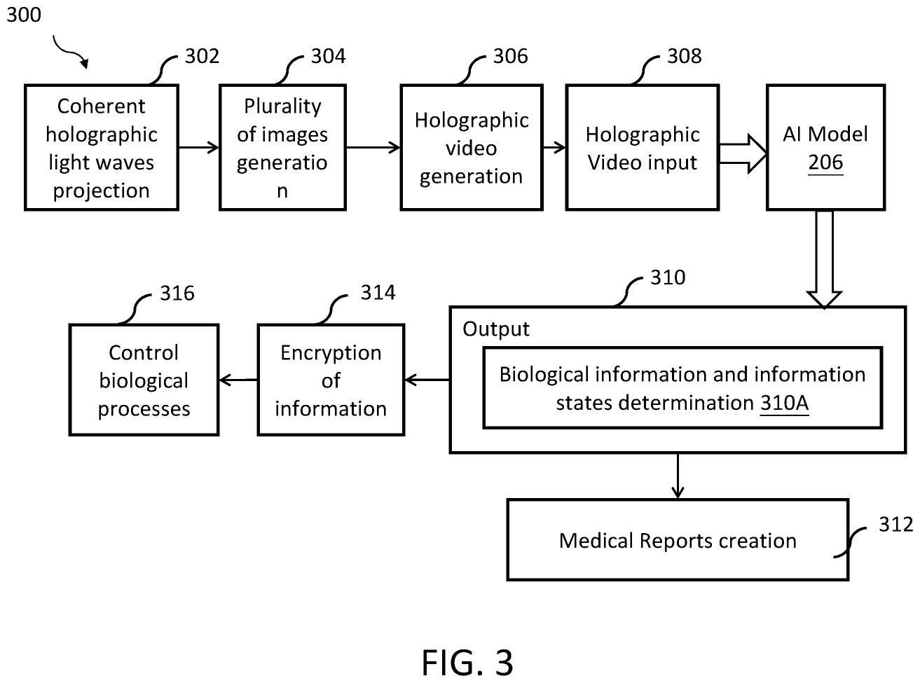

Referring now to FIG. 3, a flow diagram 300 of method steps for determining biological information of the body of the user 116 and the information states of the nervous system of the user 116 is illustrated, in accordance with an embodiment. The method steps are explained with reference to the components of FIGS. 1 and 2. The block diagram 300 may include steps 302 to 316. It will be understood that each step of the block diagram 300 may be implemented by various means, such as hardware, firmware, processor, circuitry, and/or other communication devices associated with execution of software including one or more computer program instructions.

At a step 302, the method comprises projection of the coherent holographic light waves. The coherent holographic light waves are projected on the at least one portion of the body of the user 116 by use of the coherent light source 102 with facilitation of the projecting module 204. In some embodiments, the at least one portion of the body of the user 116 may be associated with at least one of the organ of the user 116, the soft tissue of the user 116, the bone of the user 116, or the brain of the user 116. For example, the organ of the user 116 may be a heart of the user 116, a kidney of the user 116, a liver of the user 116, a pancreas of the user 116, and the like. The soft tissue of the user 116 may be for example, muscles, tendons, ligaments and/or fascia in the body of the user 116. The bone of the user 116 may be any of the 206 bones in the body of the user 116.

In an example, the user 116 may be a patient who may need medical assistance. In another example, the user 116 may be an individual with at least one disability such as Parkinson's, Alzheimer's, Amyotrophic lateral sclerosis (ALS), paralysis, post-coma unresponsiveness, and the like. It may be noted that the system 106 may be utilized by user 116 in non-medical conditions, such as enhancing of attentiveness while driving.

At a step 304, the method may further include generation of the plurality of images of the at least one portion of the body of the user 116. The coherent holographic light waves projected by the coherent light source 102 may be utilized to generate the plurality of images of the at least one portion of the body of the user 116. The generated plurality of images may be utilized to determine the biological and health conditions inside the organisms, for example, the user 116. In an embodiment, the generated plurality of images may be displayed on the display 110. The plurality of images may be utilized by, for example, the medical health professional, or the user 116.

At a step 306, the method may further include generation of the holographic video. In some embodiments, the processor 202 may be configured to generate the holographic video. In an embodiment, the holographic video may be generated by the holographic image generation module 206 by use of the plurality of images. The holographic video may indicate the logical and health conditions inside the organisms, for example, the user 116.

At a step 308, the holographic video may be input to the AI model 208. In an embodiment, the generated plurality of images may be input to the AI model 208. The AI model 208 may be trained to determine the at least one of the biological information of the body of the user 116, or the information states of the nervous system associated with the user 116, based on the input holographic video. The AI model 208 may be configured to analyze the holographic video provided as the input to determine the to provide an output the biological information of the body of the user 116, or the information states of the nervous system associated with the user 116.

At a step 310, the AI model 208 may be configured to provide an output. The output corresponds to determined biological information and information states 310A associated with the user 116. The biological information of the body of the user 116 is associated with at least one of a probability of occurrence of stroke, information related to heart disease, information related to asthma, or other health conditions of the user 116.

In an exemplary scenario, the processor 202 may project the brain of the user 116 with the coherent holographic light waves by use of the coherent light source 102. The processor 202 may further generate the holographic video based on the projection of the coherent holographic light waves to the brain of the user 116. For example, the holographic video may depict tissue damage in the brain of the user 116 that may indicate one or more symptoms of the stroke. The AI model 208 may analyze the holographic video to determine the probability of occurrence of stroke in the user 116.

In another exemplary scenario, the processor 202 may project the organ, such as the heart of the user 116 with the coherent holographic light waves by use of the coherent light source 102. The processor 202 may further generate the holographic video based on the projection of the coherent holographic light waves to the heart of the user 116. For example, the holographic video may depict blockage in arteries of the heart of the user 116 that may indicate health issue related to the heart of the user 116. The AI model 208 may analyze the holographic video to determine the information related to heart disease in the user 116. Moreover, the other health conditions of the user 116 included in the biological information may correspond to diabetes, infections, electrolyte imbalance or hormone imbalance in the user 116.

In addition, the information states of the nervous system of the user 116 may be associated with at least one of a mental state, thoughts, an emotion, an intention, motor actions, a speech, a dream, a blood flow, a neural activity, a molecular activity, or pathological information related to the user 116. In an embodiment, the mental state may correspond to an autopilot state, a critical state, a thinking state, and an engaged state. Examples of the emotions may include, but are not limited to, sadness, happiness, fear, anger, surprise and disgust. The desired motor actions, such as walking, and running may depict that the nervous system, the muscles, and the brain of the user 116 may be working in sync with each other. The information related to the neural activity in the brain of the user 116 may indicate neurodevelopmental disorders in the user 116, if any.

At a step 312, the method may include creating medical reports associated with the user 116 based on the determined biological information and the information states 312 of the user 116. The medical reports may include details of the determined information and the information states 312 of the user 116. For example, medical reports may indicate presence of blockage in the arteries of the user 116. The medical reports may be used by one or more healthcare professionals to provide treatment to the user 116. In an embodiment, the medical reports of the user 116 may be utilized by the user 116 to provide health related guidance and self-improvement for the user 116. The medical reports may be utilized by the user 116 to take necessary actions, such as precautions in order to avoid health related hazards. The necessary actions include but may not be limited to going for a walk, taking rest, exercising, and taking medicine on time.

At a step 314, the method may further include encryption of the determined biological information the information states 312 of the user 116. The determined at least one of the biological information of the body of the user 116, or the information states of the nervous system associated with the user 116 may be secured based on one or more encryption techniques. In addition, the at least one of the biological information or the information states of the nervous system are secured in compliance with at least one of Health Insurance Portability and Accountability Act (HIPAA) or General Data Protection Regulation (GDPR). Thus, the system 106 may ensure that the recorded information, such as the plurality of images may be secured and a misuse of the plurality of images may be avoided. Furthermore, the created medical reports of the user 116 may be encrypted, such that the medical reports of the user 116 may only be accessed by the user 116 or any authorized personnel, such as the one or more healthcare professionals.

At a step 316, the method may further include controlling of the one or more biological processes within the user 116. In an embodiment, the processor 202 may be configured to transmit the one or more signals to at least the neurons in the body of the user 116 based on the analyzed plurality of images for control of the one or more biological processes associated with the user 116.

In an exemplary scenario, the analyzed holographic video may determine presence of stress and anxiety in the user 116. Based on the determination, the processor 202 may transmit the one or more signals that may correspond to stress and anxiety stress and anxiety busting activities through the communication interface 214 to control the stress level and the anxiety in the user 116. Thus, in such a manner, the processor 202 may be configured to control the one or more biological processes within the user 116.

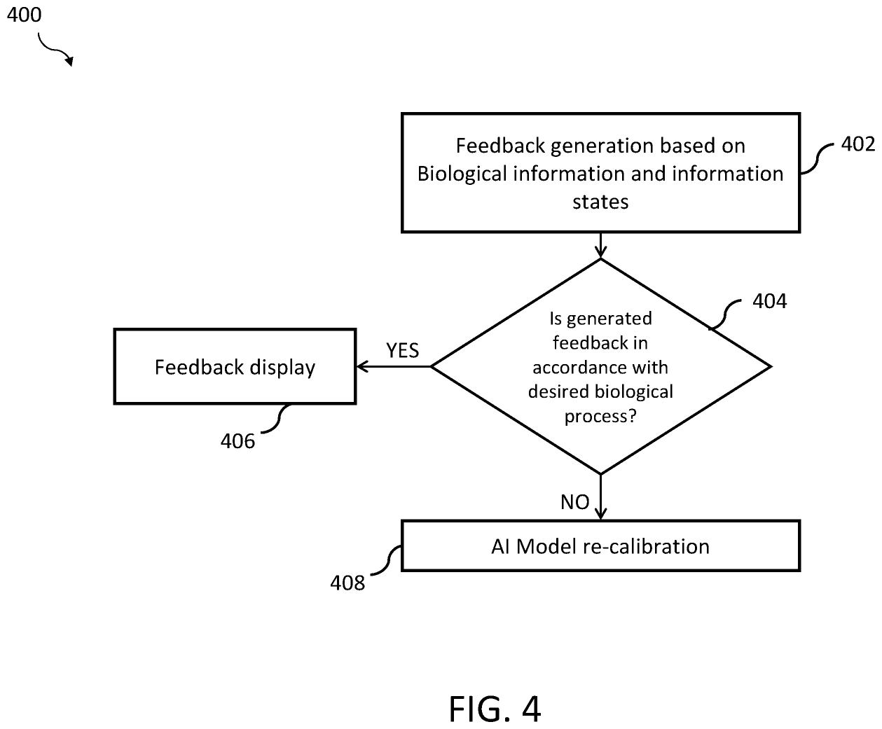

In an embodiment, the processor 202 may be further configured to measure the one or more biological processes within the user 116 after the transmission of the one or more signals. Based on the measured one or more biological processes within the user 116, the processor 202 may be further configured to generate feedback. Details of the generation of the feedback are further provided, for example, in FIG. 4.

In some embodiments, the method may further include controlling the interference of the coherent holographic light waves transmitted as the one or more signals, to transfer energy associated with the coherent holographic light waves projected to the at least one portion of the body of the user 116 at a particular time. In general, interference of waves is the phenomenon that occurs when two waves meet while travelling along the same medium to form a resultant wave of greater, lower, or same amplitude. The interference of the coherent holographic light waves may be controlled to transfer a sufficient or a required amount of energy to the at least one portion of the user 116 at the particular time to determine the biological information and the information states of a region of interest of the at least one portion of the user 116. For example, the interference of the coherent holographic light waves is controlled to transfer the required amount of energy projected to cerebellum of the brain of the user 116 to determine the biological information and the information state of the cerebellum.

In some embodiments, the method may further include controlling an interference of the coherent holographic light waves transmitted as the one or more signals, to dampen energy associated with the coherent holographic light waves projected to the at least one portion of the body of the user 116 at a particular time. The interference of the coherent holographic light waves is controlled to dampen energy projected to the at least one portion of the user 116 at the particular time to acquire the biological information and the information states of, for example, a delicate region of interest in the at least one portion of the user 116. In general, dampening energy of the coherent holographic waves corresponds to reduction in energy per unit volume that causes amplitude of the coherent holographic waves to decrease with increasing displacement.

The method terminates at step 316.

The method is not limited to the above-mentioned steps. For example, if a desired output of the determined biological information and the information states 312 is not achieved by the user 116 or if the generated feedback is not in accordance with the desired output (such as a desired control of the biological process within the user 116), the method may include re-calibration of the AI model 208. The re-calibration of the AI model 208 is further explained in FIG. 4.

Referring now to FIG. 4, a flow chart 400 for displaying feedback associated with informatics and biological processes is illustrated, in accordance with an embodiment. The flow chart 400 is explained in reference with the components of FIGS. 1, 2 and 3. The flow chart 400 may include steps 402 to 408. It will be understood that each step of the flow chart 400 may be implemented by various means, such as hardware, firmware, processor, circuitry, and/or other communication devices associated with execution of software including one or more computer program instructions.

At a step 402, the method may include generation of the feedback. In some embodiments, the processor 202 may be configured to measure the one or more biological processes within the user 116 after the transmission of the one or more signals. In an example, the processor 202 may be further configured to measure the biological information of the body of the user 116 and the information states of the nervous system of the user 116 again after the transmission of the one or more signals to the body of the user 116.

In an exemplary scenario, the user 116 may need to increase a level of concentration. The processor 202 may project at least the brain of the user 116 with coherent holographic light waves. The processor 202 may further generate the holographic video that may indicate an initial level of concentration of the user 116. The processor 202 may transmit the one or more signals to increase the level of concentration of the user 116. The processor 202 may further generate the holographic video that may indicate an updated level of concentration of the user 116, after transmission of the one or more signals to increase the level of concentration of the user 116. The holographic video that may indicate the updated level of concentration of the user 116 may be the generated feedback by the processor 202.

At a step 404, the method may include checking if the feedback is in accordance with the desired biological process in the body of the user 116. In one or more embodiments, the processor 202 may be configured to check if the generated feedback is in accordance with the biological process in the body of the user 116. For example, the generated feedback may include the updated level of concentration as “low” or “high”. The processor 202 may check if the generated feedback, i.e., the low level of concentration or the high level of concentration or is required as the desired biological process.

At a step 406, the method may further include displaying of the feedback to the user 116, when the generated feedback is in accordance with the desired output. In an embodiment, the generated feedback is displayed to the user 116 on the display 110 via the electronic device 108. In an exemplary scenario, the generated feedback may depict that the updated level of concentration is “high”. The generated feedback in such a case same as the desired effect of “increase in the level of concentration”. Thus, the processor 202 may display the generated feedback to the user 116 on the display 110.

At a step 408, the method may further include re-calibration of the AI model 208, when the generated feedback is not in accordance with the desired output. In an exemplary scenario, the generated feedback may depict that the updated level of concentration is “low”. The generated feedback in such a case is different than the desired effect of “increase in the level of concentration”. Thus, the processor 202 may provide the generated feedback to the AI model 208 as the input. The processor 202 may further re-calibrate the AI model 208 to produce the desired output. For example, the generated holographic video may again be output by the AI model 208. The processor 202 may generate the one or signals to achieve the desired effect of increase in the level of concentration based on the generated holographic video. Therefore, the re-calibration of the AI model 208 iteratively for the learning, such as the deep learning may be enabled by the processor 202. In such a manner, the processor 202 may enable accurate, non-invasive and safe control of the one or more biological processes associated with the user 116.

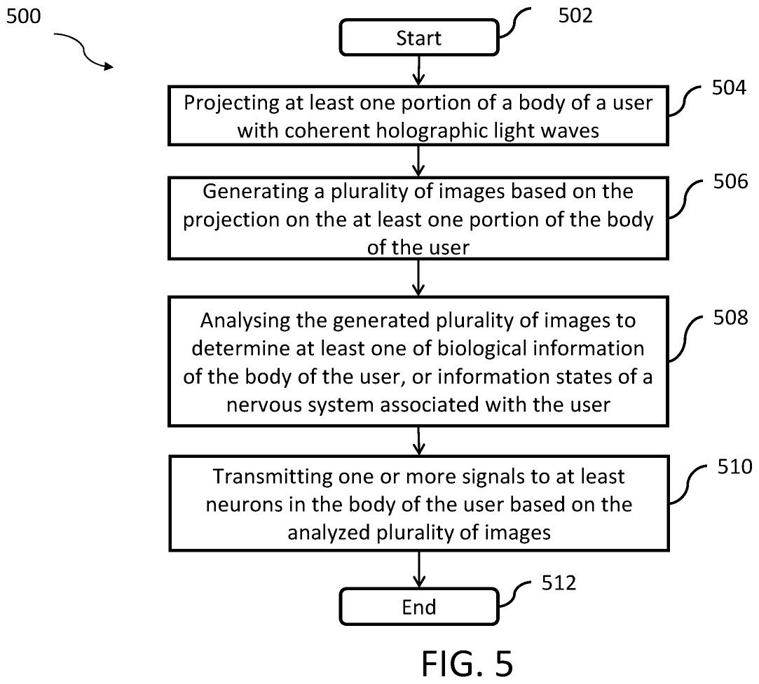

Referring now to FIG. 5, a flow chart depicting a method 500 for controlling informatics and biological processes is illustrated, in accordance with an embodiment. The flow chart 500 is explained in reference with the components of FIGS. 1, 2, 3 and 4. The flow chart 500 may include steps 502 to 512. It will be understood that each step of the flow chart 500 may be implemented by various means, such as hardware, firmware, processor, circuitry, and/or other communication devices associated with execution of software including one or more computer program instructions. The method 500 initiates at step 502.

At a step 504, the method 500 includes projecting the at least one portion of the body of a user 116 with the coherent holographic light waves through the coherent light source 102. The coherent light source 102 projects the coherent holographic light waves on the at least one portion of the body of the user 116. In general, coherent light source emits a light wave that has same frequency, and wavelength and a constant phase difference. The coherent light source 102 forms sustained interference patterns when superimposition of light waves occurs. In an embodiment, the coherent light source 102 includes but may not be limited to the laser light. The highly coherent light from laser is parallel, monochromatic and has unbroken wave chains. In an embodiment, the coherent light source 102 projects the coherent holographic light waves on the at least one portion of the body of the user with facilitation of the light projection module 202.

The light projection module 202 is configured to project the at least one portion of the body of the user 116 with the coherent holographic light waves by using the coherent light source 102. The light projection module 202 uses different lights of different wavelengths and frequencies for different portions of the body of the user 116. For example, if retina of an eye of the user 116 is being used as a pathway to send a light wave as an input, then the light projection module 202 is configured to utilize visible range of frequencies of the light wave. In another example, if a different tissue is targeted, such as some cancer cells in leg portion of the user 116, the light projection module 202 may use some other waveforms of the coherent holographic light waves of different wavelengths and frequencies. Once the light wave reaches the tissue, generation of the plurality of images starts.

At a step 506, the method 500 includes generating the plurality of images based on the projection of the at least one portion of the body of the user 116. Each of the plurality of images is generated with facilitation of the holographic image generation module 206. The holographic image generation module 206 performs utilization of the coherent holographic light waves projected by the coherent light source 102 for generating the plurality of images of the at least one portion of the body of the user 116. In addition, the holographic image generating module 206 is configured to generate the holographic video based on the plurality of images.

At a step 508, the method 500 includes analysis of the generated plurality of images to determine at least one of biological information of the body of the user 116, or information states of the nervous system associated with the user 116. The analysis of the generated plurality of images and the holographic video is performed by the AI model 208. The AI model 208 utilizes the generated plurality of images and the generated holographic video for determining at least one of the biological information of the body of the user 116, or the information states of the nervous system associated with the user 116. The biological information of the body of the user 116 is associated with at least one of a probability of occurrence of stroke, information related to heart disease, information related to asthma, or other health conditions of the user 116. In addition, the information states of the nervous system of the user 116 is associated with at least one of the mental state, thoughts, the emotion, the intention, motor actions, the speech, the dream, the blood flow, the neural activity, the molecular activity, or pathological information related to the user 116. In an example, the AI model 208 is configured to read intentions, or thoughts of the user 116 based on the analyzed plurality of images and the holographic video. Further, the AI model 208 allows interaction with the user 116 based on the read intentions or thoughts of the user 116 with facilitation of the communication interface 214 of the system 106.

At step 510, the method 500 includes transmitting one or more signals to at least neurons in the body of the user 116 based on the analyzed plurality of images. The one or more signals are transmitted to the at least neurons in the body of the user 116 with facilitation of the transmitting module 210. The transmitting module 210 is configured to transmit the one or more signals to at least neurons in the body of the user 116 based on the analyzed plurality of images for controlling informatics and the one or more biological processes associated with the user 116. The one or more signals are transmitted to the central nervous system of the user 116 to control the one or more biological processes within the user 116. In an example, the one or more signals are used to write information in the brain of the user 116. The one or more signals are transmitted based on the projection of the coherent holographic light waves. It should be noted that transmitting the one or more signals to the at least neurons in the body of the user 116 may further include the interference of the coherent holographic light waves transmitted as the one or more signals, to transfer energy associated with the coherent holographic light waves projected on the at least one portion of the body of the user 116 at a particular time. In addition, transmitting the one or more signals to the at least neurons in the body of the user 116 may further include controlling the interference of the coherent holographic light waves transmitted as the one or more signals, to dampen energy associated with the coherent holographic light waves projected to the at least one portion of the body of the user at a particular time.

At step 512, the method 500 terminates.

It will be appreciated as the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

The disclosed methods and systems may be implemented on a conventional or a general-purpose computer system, such as a personal computer (PC) or server computer. It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

The disclosed methods and systems may be implemented on a conventional or a general-purpose computer system, such as a personal computer (PC) or server computer.

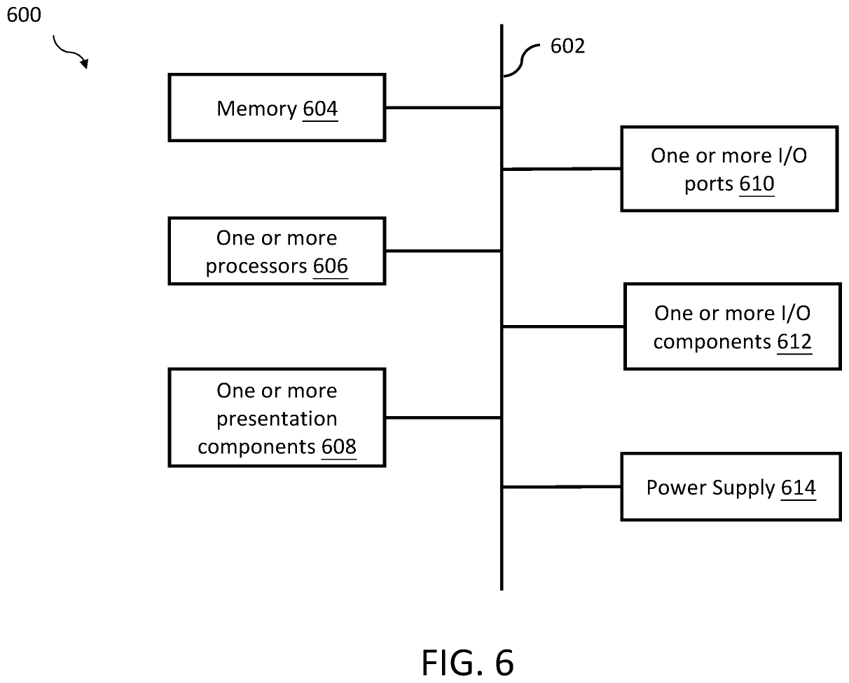

Referring now to FIG. 6, a block diagram of an exemplary computer system 600 for implementing various embodiments is illustrated. The computer system 600 includes a bus 602 that directly or indirectly couples the following devices: memory 604, one or more processors 606, one or more presentation components 608, one or more input/output (I/O) ports 610, one or more input/output components 612, and an illustrative power supply 614. The bus 602 represents what may be one or more busses (such as an address bus, data bus, or combination thereof). Although the various blocks of FIG. 6 are shown with lines for the sake of clarity, in reality, delineating various components is not so clear, and metaphorically, the lines would more accurately be grey and fuzzy. For example, one may consider a presentation component such as a display device to be an I/O component. It may be understood that the diagram of FIG. 6 is merely illustrative of an exemplary system 106 that can be used in connection with one or more embodiments of the present invention. The distinction is not made between such categories as “server,” “laptop,” “hand-held device,” etc., as all are contemplated within the scope of FIG. 6 and reference to “a system 106 of FIG. 1.”

The computer system 600 typically includes a variety of computer-readable media. The computer-readable media can be any available media that can be accessed by the computer system 600 and includes both volatile and nonvolatile media, removable and non-removable media. By way of example, and not limitation, the computer-readable media may comprise computer readable storage media and communication media. The computer readable storage media includes volatile and nonvolatile, removable and non-removable media implemented in any method or technology for storage of information such as computer-readable instructions, data structures, program modules or other data.

The computer-readable storage media with memory 604 includes, but is not limited to, non-transitory computer readable media that stores program code and/or data for longer periods of time such as secondary or persistent long term storage, like RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computer system 600. The computer-readable storage media associated with the memory 604 and/or other computer-readable media described herein can be considered computer readable storage media for example, or a tangible storage device. By way of example, and not limitation, communication media includes wired media such as a wired network or direct-wired connection, and wireless media such as acoustic, RF, infrared and other wireless media. Combinations of any of the above should also be included within the scope of computer-readable media. The computer system 600 includes one or more processors that read data from various entities such as the memory 604 or I/O components 612. The one or more presentation components 608 present data indications to a user or other device. Exemplary presentation components include a display device, speaker, printing component, vibrating component, etc. The one or more I/O ports 610 allow the computer system 600 to be logically coupled to other devices including the one or more I/O components 612, some of which may be built in. Illustrative components include a microphone, joystick, game pad, satellite dish, scanner, printer, wireless device, etc.

The above-described embodiments of the present disclosure may be implemented in any of numerous ways. For example, the embodiments may be implemented using hardware, software or a combination thereof. When implemented in software, the software code may be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. Typically, the functionality of the program modules may be combined or distributed as desired in various embodiments.

Also, the embodiments of the present disclosure may be embodied as a method, of which an example has been provided. The acts performed as part of the method may be ordered in any suitable way. Accordingly, embodiments may be constructed in which acts are performed in an order different than illustrated, which may include performing some acts concurrently, even though shown as sequential acts in illustrative embodiments. Therefore, it is the object of the appended claims to cover all such variations and modifications as come within the true spirit and scope of the present disclosure.

Although the present disclosure has been described with reference to certain preferred embodiments, it is to be understood that various other adaptations and modifications can be made within the spirit and scope of the present disclosure. Therefore, it is the aspect of the append claims to cover all such variations and modifications as come within the true spirit and scope of the present disclosure.

What is claimed is:

1. A method for controlling informatics and biological processes comprising:
projecting at least one portion of a body of a user with coherent holographic light waves by use of a coherent light source;
generating a plurality of images based on the projection on the at least one portion of the body of the user with the coherent holographic light waves;
analyzing the generated plurality of images to determine at least one of: biological information of the body of the user, or information states of a nervous system associated with the user; and
transmitting one or more signals to at least neurons in the body of the user based on the analyzed plurality of images for control of one or more biological processes associated with the user, wherein the one or more signals are transmitted based on the projection of the coherent holographic light waves.

2. The method of claim 1, wherein the at least one portion of the body is associated with at least one of: an organ of the user, a soft tissue of the user, a bone of the user, or a brain of the user.

3. The method of claim 1, further comprising:
generating a holographic video based on the plurality of images; and
analyzing the generated holographic video to determine at least one of: the biological information of the body of the user, or the information states of the nervous system associated with the user.

4. The method of claim 1, wherein the determined biological information of the body of the user is associated with at least one of: a probability of occurrence of stroke, information related to heart disease, information related to asthma, or other health conditions of the user.

5. The method of claim 1, wherein the determined information states of the nervous system of the user is associated with at least one of: a mental state, thoughts, an emotion, an intention, motor actions, a speech, a dream, a blood flow, a neural activity, a molecular activity, or pathological information related to the user.

6. The method of claim 1, further comprising creating medical reports associated with the user based on at least one of: the determined biological information of the body of the user, and the information states of the nervous system of the user.

7. The method of claim 1, further comprising utilizing an artificial intelligence (AI) model for analysis of the generated plurality of images to determine the at least one of the biological information of the body of the user, or the information states of the nervous system associated with the user.

8. The method of claim 1, further comprising controlling an interference of the coherent holographic light waves transmitted as the one or more signals, to transfer energy associated with the coherent holographic light waves projected on the at least one portion of the body of the user at a particular time.

9. The method of claim 1, further comprising controlling an interference of the coherent holographic light waves transmitted as the one or more signals, to dampen energy associated with the coherent holographic light waves projected to the at least one portion of the body of the user at a particular time.

10. The method of claim 1, further comprising:
generating feedback associated with the determined at least one of the biological information of the body of the user, or the information states of the nervous system associated with the user; and
displaying the generated feedback to the user.

11. The method of claim 10, further comprising re-calibrating an AI model utilized for analysis of the generated plurality of images, based on the generated feedback.

12. The method of claim 1, further comprising securing the determined at least one of the biological information of the body of the user, or the information states of the nervous system associated with the user, based one or more encryption techniques, and
wherein at least the biological information and the information states of the nervous system are secured in compliance with at least one of: Health Insurance Portability and Accountability Act (HIPAA) or General Data Protection Regulation (GDPR).

13. A system for controlling informatics and biological processes, the system comprising
a processor; and
a memory communicatively coupled to the processor, wherein the memory stores a plurality of processor-executable instructions which upon execution by the processor cause the processor to:
project at least one portion of a body of a user with coherent holographic light waves by use of a coherent light source;
generate a plurality of images based on the projection of the at least one portion of the body of the user with the holographic light waves;
analyze the generated plurality of images to determine at least one of: biological information of the body of the user, or information states of a nervous system associated with the user; and
transmit one or more signals to at least neurons in the body of the user based on the analyzed plurality of images for control of one or more biological processes associated with the user, wherein the one or more signals are transmitted based on the projection of the coherent holographic light waves.

14. The system of claim 13, wherein the plurality of processor-executable instructions, upon execution by the processor, further cause the processor to:
generate a holographic video based on the plurality of images; and
analyze the generated holographic video to determine at least one of: the biological information of the body of the user, or the information states of the nervous system associated with the user.

15. The system of claim 13, wherein the determined biological information of the body of the user is associated with at least one of: a probability of occurrence of stroke, information related to heart disease, information related to asthma, or other health conditions of the user.

16. The system of claim 13, wherein the determined information states of the nervous system of the user is associated with at least one of: a mental state, thoughts, an emotion, an intention, motor actions, a speech, a dream, a blood flow, a neural activity, a molecular activity, or pathological information related to the user.

17. The system of claim 13, wherein the plurality of processor-executable instructions, upon execution by the processor, further cause the processor to create medical reports associated with the user based on at least one of: the determined biological information of the body of the user, and the information states of the nervous system of the user.

18. The system of claim 13, wherein the plurality of processor-executable instructions, upon execution by the processor, further cause the processor to utilize an artificial intelligence (AI) model for analysis of the generated plurality of images to determine the at least one of the biological information of the body of the user, or the information states of the nervous system associated with the user.

19. The system of claim 13, wherein the plurality of processor-executable instructions, upon execution by the processor, further cause the processor to:
generate feedback associated with the determined at least one of the biological information of the body of the user, or the information states of the nervous system associated with the user; and
display the generated feedback to the user.

20. A computer programmable product comprising a non-transitory computer readable medium having stored thereon computer executable instruction which when executed by one or more processors, cause the one or more processors to carry out operations for controlling informatics and biological processes, the operations comprising:
projecting at least one portion of a body of a user with coherent holographic light waves by use of a coherent light source;
generating a plurality of images based on the projection of the at least one portion of the body of the user with the holographic light waves;
analyzing the generated plurality of images to determine at least one of: biological information of the body of the user, or information states of a nervous system associated with the user; and
transmitting one or more signals to at least neurons in the body of the user based on the analyzed plurality of images for control of one or more biological processes associated with the user, wherein the one or more signals are transmitted based on the projection of the coherent holographic light waves.
